(19) United States
(12) Patent Application Publication
(10) Pub. No.: US 2025/0288419 A1
(43) Pub. Date: Sep. 18, 2025

(54) PENIS ENLARGEMENT IMPLANT AND PREPARATION METHOD

(71) Applicant: SOUTH CHINA UNIVERSITY OF TECHNOLOGY, Guangzhou (CN)

(72) Inventors: Xuetao SHI, Guangzhou (CN); Rui ZHENG, Guangzhou (CN); Muyuan CHAI, Guangzhou (CN)

(73) Assignee: SOUTH CHINA UNIVERSITY OF TECHNOLOGY, Guangzhou (CN)

(21) Appl. No.: 19/060,827

(22) Filed: Feb. 24, 2025

(30) Foreign Application Priority Data
Mar. 13, 2024 (CN) 202410286753.7

(51) Int. Cl. A61F 2/26

(52) U.S. Cl. CPC A61F 2/26; A61F 2240/001; A61F 2250/0058

(57) ABSTRACT

The present disclosure discloses a penis enlargement implant and a preparation method, and relates to the technical field of medical instruments. The preparation method of the penis enlargement implant comprises: loading a polyvinyl alcohol solution into a mold, the mold having a negative Poisson's ratio structure; performing at least three freeze-thaw cycles on the mold and the polyvinyl alcohol solution to obtain a hydrogel sample; soaking the hydrogel sample in a lye for a preset time period, and then washing to remove impurities to obtain the penis enlargement implant. The preparation method of the present disclosure uses a biocompatible hydrogel as a matrix material, which is not susceptible to degradation or localized fibrosis after implantation, and can be used for a long time. In addition, the penis enlargement implant obtained by the preparation method has an elongation at break of 200-400%.

### CROSS-REFERENCE TO RELATED APPLICATIONS

This application claims priority to Chinese Application No. 202410286753.7, filed on Mar. 13, 2024, the entire contents of which are hereby incorporated by reference.

### TECHNICAL FIELD

The present disclosure relates to the technical field of medical instruments, and in particular to a penis enlargement implant and a preparation method.

### BACKGROUND

Throughout history, penis size has been a major concern for men, and a larger penis is often considered a symbol of masculinity. In recent times, as “locker room syndrome” caused by psychological factors, congenital micropenis syndrome, the penis defect or deformity resulted from acquired penile injuries, etc., people have a more extensive demand for penile plastic surgery, especially penis enlargement.

With the development of science and technology, the current implementation of penis enlargement mainly includes non-surgical and surgical solutions. The non-surgical solutions mainly include vacuum device traction, filler injection, etc. The vacuum device traction has poor efficacy and causes pain to patients. The filler injection is generally implemented by injecting hyaluronic acid, polylactic acid, and other common hypodermic injections, which are susceptible to degradation after a certain period of time (3-6 months), making it difficult to maintain the effect of enlargement for a long time. The surgical solutions can be implemented by surgically loosening suspensory ligaments, etc. However, some improper operations may lead to penile erection deformity. Some patients choose to directly implant a thickening material to achieve long-lasting enlargement. This material mainly includes allogeneic dermal matrix, autologous fat, etc. Implants of biological origin are easy to degrade, while artificial material implants that are not biocompatible may lead to infection. Penuma, a medical silicone rubber penile implant currently on the market, has attracted widespread attention from clinicians, but the implant still suffers from a large difference in stiffness and poor compatibility with the human penis, and other problems, which may cause edema and discomfort to users. The use with polypropylene plastic for fixation may also lead to complications such as hematoma, hyperplastic scarring, etc. In addition, this type of product has poor customization, high price, and other problems.

The change in the implant during penile erection is critical in terms of long-term use. When the male penis is stimulated, the blood vessels dilate and blood rapidly fills the corpus cavernosum of the penis, ultimately leading to the erection and hardening. During this process, the penis lengthens with a corresponding increase in the girth (i.e., thickening). Most injectable materials are not comparable to the strength of the penis during erection due to the poor mechanical properties, and implantable materials are not able to change along with the changes in the penis, which may lead to implant displacement, erection pain, and other problems. Many materials are designed ignoring the anisotropic changes in the penis. Therefore, it is significant to develop a penis enlargement implant with a high degree of compatibility and excellent compliance, and a preparation method thereof.

### SUMMARY

Some embodiments of the present disclosure provide a preparation method of a penis enlargement implant, comprising: loading a polyvinyl alcohol solution into a mold, the mold having a negative Poisson's ratio structure; performing at least three freeze-thaw cycles on the mold and the polyvinyl alcohol solution to obtain a hydrogel sample; and soaking the hydrogel sample in a lye for a preset time period, and then washing to remove impurities to obtain the penis enlargement implant.

In some embodiments, a molecular weight of polyvinyl alcohol in the polyvinyl alcohol solution may be in a range of 10000 Da-196000 Da, and an alcoholysis degree may be in a range of 90%-98%.

In some embodiments, a concentration of the polyvinyl alcohol solution may be in a range of 12.5 wt %-17.5 wt %.

In some embodiments, the lye may be a NaOH solution or a KOH solution, and a concentration of the NaOH solution or a KOH solution may be in a range of 17.5 wt %-22.5 wt %.

In some embodiments, an elongation at break of the hydrogel sample may be greater than or equal to 200%, a Young's modulus of the hydrogel sample may be greater than or equal to 1 MPa, and a compression modulus of the hydrogel sample may be greater than or equal to 200 kPa.

In some embodiments, the performing at least three freeze-thaw cycles on the mold and the polyvinyl alcohol solution to obtain a hydrogel sample may include: freezing the mold and the polyvinyl alcohol solution at a temperature from −30° C. to −15° C. for 6 h-12 h, then thawing at a temperature from 10° C. to 40° C. for 4 h-6 h, and cycling the freezing and the thawing for 3-5 times to obtain the hydrogel sample.

In some embodiments, the soaking the hydrogel sample in a lye for a preset time period, and then washing to remove impurities to obtain the penis enlargement implant may include: placing the hydrogel sample in the lye and stirring for 20 h-30 h, and then placing the hydrogel sample in water and stirring and washing for 22 h-26 h to obtain the penis enlargement implant, during the stirring and the washing, changing the water every 4 h-8 h.

In some embodiments, the mold may include a border and a plurality of unit elements periodically arranged. The plurality of unit elements may include at least one of a concave hexagonal unit element, a stellate unit element, a bend-induced unit element, and a chiral unit element.

In some embodiments, each of the plurality of unit elements may be the concave hexagonal unit element, and each of the plurality of unit elements may include a bottom wall, a top wall, a first bottom sloping wall, a second bottom sloping wall, a first top sloping wall, and a second top sloping wall. The bottom wall and the top wall may be arranged parallel to each other. The first bottom sloping wall and the second bottom sloping wall may be arranged on two sides of the bottom wall, and a first preset angle may be formed between the first bottom sloping wall and the second bottom sloping wall and the bottom wall. The first top sloping wall and the second top sloping wall may be arranged on two sides of the top wall, and a second preset angle may be formed between the first top sloping wall and the second top sloping wall and the top wall. One end of the first bottom sloping wall may be connected with the bottom wall, the other end of the first bottom sloping wall may be connected with the first top sloping wall. One end of the second bottom sloping wall may be connected with the bottom wall, the other end of the second bottom sloping wall may be connected with the second top sloping wall. The first bottom sloping wall may be parallel to the second top sloping wall. The second bottom sloping wall may be parallel to the first top sloping wall.

In some embodiments, a distance between the bottom wall and the top wall, and a height of the bottom wall may satisfy the following equation: tana ≥w/h; where w denotes the distance between the bottom wall and the top wall, h denotes the height of the bottom wall, and a denotes the first preset angle.

In some embodiments, the first preset angle and the second preset angle may be the same; the first preset angle may be in a range of 20°-60°, and the second preset angle may be in a range of 20°-60°.

In some embodiments, the distance between the top wall and the bottom wall may be in a range of 1.8 mm-3.5 mm.

In some embodiments, a height of the bottom wall and a height of the top wall may be the same; the height of the bottom wall may be in a range of 1 mm-4 mm, and the height of the top wall may be in a range of 1 mm-4 mm.

In some embodiments, a thickness of the bottom wall, a thickness of the top wall, a thickness of the first bottom sloping wall, a thickness of the second bottom sloping wall, a thickness of the first top sloping wall, and a thickness of the second top sloping wall may be the same; the thickness of the bottom wall may be in a range of 0.6 mm-1.2 mm, the thickness of the top wall may be in a range of 0.6 mm-1.2 mm, the thickness of the first bottom sloping wall may be in a range of 0.6 mm-1.2 mm, the thickness of the second bottom sloping wall may be in a range of 0.6 mm-1.2 mm, the thickness of the first top sloping wall may be in a range of 0.6 mm-1.2 mm, and the thickness of the second top sloping wall may be in a range of 0.6 mm-1.2 mm.

In some embodiments, each of the plurality of unit elements may be the stellate unit element. Each of the plurality of unit elements may be enclosed by three arrowhead units. Apex angles of the three arrowhead units may be the same. The apex angle of each of the three arrowhead units may be in a range of 10°-30°; and/or a distance between adjacent unit elements of the plurality of unit elements may be in a range of 0.2 mm-1.0 mm.

In some embodiments, each of the plurality of unit elements may be the bend-induced unit element, and each of the plurality of unit elements may include a first unit and a second unit. The first unit may include a first ellipsoidal unit of which a long axis is set vertically and a second ellipsoidal unit of which a long axis is set horizontally. The second unit may include a third ellipsoidal unit of which a long axis is set vertically and a fourth ellipsoidal unit of which a long axis is set horizontally. The long axis of the first ellipsoidal unit and the long axis of the fourth ellipsoidal unit may be parallel to each other. The long axis of the second ellipsoidal unit and the long axis of the third ellipsoidal unit may be parallel to each other. The long axis of the first ellipsoidal unit may be coaxial with a short axis of the third ellipsoidal unit, and a short axis of the second ellipsoidal unit may be coaxial with the long axis of the fourth ellipsoidal unit.

In some embodiments, a length of the long axis of the first ellipsoidal unit, a length of the long axis of the second ellipsoidal unit, a length of the long axis of the third ellipsoidal unit, and a length of the long axis of the fourth ellipsoidal unit may be the same, and a length of the short axis of the first ellipsoidal unit, a length of the short axis of the second ellipsoidal unit, a length of the short axis of the third ellipsoidal unit, and a length of the short axis of the fourth ellipsoidal unit may be the same; the length of the long axis may be in a range of 1.5 mm-5.5 mm, and the length of the short axis may be in a range of 0.5 mm-3.5 mm; and/or a distance between the first ellipsoidal unit and the second ellipsoidal unit may be the same as a distance between the first ellipsoidal unit and the third ellipsoidal unit; the distance between the first ellipsoidal unit and the second ellipsoidal unit may be in a range of 0.1 mm-1.5 mm, and the distance between the first ellipsoidal unit and the third ellipsoidal unit may be in a range of 0.1 mm-1.5 mm.

In some embodiments, each of the plurality of unit elements may be the chiral structural unit element, and each of the plurality of unit elements may include a unit shaped like a Chinese character “wan”. The unit shaped like a Chinese character “wan” may include a first wall and a second wall which are perpendicular to each other. Each of two ends of the first wall may be provided with a first bending portion perpendicular to the first wall, and each of two ends of the second wall may be provided with a second bending portion perpendicular to the second wall.

In some embodiments, a thickness of the first wall, a thickness of the second wall, a thickness of the first bending portion, and a thickness of the second bending portion may be the same, and the thickness of the first wall, the thickness of the second wall, the thickness of the first bending portion, and the thickness of the second bending portion may be in a range of 0.1 mm-1.5 mm, respectively; a distance between the first bending portion and the second wall may be the same as a distance between the second bending portion and the first wall; the distance between the first bending portion and the second wall may be in a range of 0.5 mm-1.5 mm, and the distance between the second bending portion and the first wall may be in a range of 0.5 mm-1.5 mm.

Some embodiments of the present disclosure further disclose a penis enlargement implant, prepared by the preparation method described above.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering indicates the same structure, wherein:

FIG. 1 is a schematic structural diagram illustrating an exemplary mold according to some embodiments of the present disclosure;

FIG. 2 is a schematic structural diagram illustrating an exemplary mold according to some embodiments of the present disclosure;

FIG. 3 is a schematic structural diagram illustrating an exemplary mold according to some embodiments of the present disclosure;

FIG. 4 is a schematic structural diagram illustrating an exemplary mold according to some embodiments of the present disclosure;

FIG. 5 is a schematic structural diagram illustrating an exemplary penis enlargement implant prepared according to Embodiment 1 of the present disclosure;

FIG. 6 is a schematic structural diagram illustrating an exemplary penis enlargement implant prepared according to Embodiment 2 of the present disclosure;

FIG. 7 is a schematic structural diagram illustrating an exemplary penis enlargement implant prepared according to Embodiment 3 of the present disclosure;

FIG. 8 is a schematic structural diagram illustrating an exemplary penis enlargement implant prepared according to Embodiment 4 of the present disclosure;

FIG. 9 is a schematic structural diagram illustrating an exemplary penis enlargement implant prepared according to Contrast Embodiment 1 of the present disclosure; and

FIG. 10 is a schematic structural diagram illustrating an exemplary penis enlargement implant prepared according to Contrast Embodiment 2 of the present disclosure.

### DETAILED DESCRIPTION

In order to more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for a person of ordinary skill in the art to apply the present disclosure to other similar scenarios in accordance with these drawings without creative labor. Unless obviously obtained from the context or the context illustrates otherwise, the same numeral in the drawings refers to the same structure or operation.

It should be understood that the terms “system,” “device,” “unit” and/or “module” used herein are a way to distinguish between different components, elements, parts, sections, or assemblies at different levels. However, the terms may be replaced by other expressions if other words accomplish the same purpose.

As shown in the present disclosure and in the claims, unless the context clearly suggests an exception, the words “one,” “a,” “an,” “one kind,” and/or “the” do not refer specifically to the singular, but may also include the plural. Generally, the terms “including” and “comprising” suggest only the inclusion of clearly identified steps and elements, however, the steps and elements that do not constitute an exclusive list, and the method or apparatus may also include other steps or elements.

The present disclosure provides a preparation method of a penis enlargement implant, comprising:

S1: loading a polyvinyl alcohol solution into a mold.

The polyvinyl alcohol solution is an aqueous solution of polyvinyl alcohol, which is prepared by dissolving the polyvinyl alcohol in water at a temperature of 80° C.-95° C. In some embodiments, a molecular weight of the polyvinyl alcohol in the polyvinyl alcohol solution may be in a range of 10000 Da-196000 Da, and an alcoholysis degree may be in a range of 90%-98%. For example, the molecular weight of the polyvinyl alcohol is in a range of 10000 Da-196000 Da, and the alcoholysis degree is in a range of 85%-99.9%. As another example, the molecular weight of the polyvinyl alcohol is in a range of 146000 Da-186000 Da, and the alcoholysis degree is in a range of 95%-99%. The molecular weight and the alcoholysis degree of the polyvinyl alcohol in the polyvinyl alcohol solution are set in this way, so that the mechanical properties (e.g., stiffness, etc.) of the polyvinyl alcohol after freeze-thaw cycles further match the performance of the penis before and after erection.

In some embodiments, a concentration of the polyvinyl alcohol solution may be in a range of 12.5 wt %-17.5 wt %. A higher polyvinyl alcohol content, while further increasing the strength, reduces the subsequent processing performance. Accordingly, the polyvinyl alcohol solution at the concentration can facilitate subsequent processing while making the resulting penis enlargement implant have appropriate strength. For example, the concentration of the polyvinyl alcohol is in a range of 13.5 wt %-16.5 wt %. As another example, the concentration of the polyvinyl alcohol is in a range of 14 wt %-16 wt %. As another example, the concentration of the polyvinyl alcohol is in a range of 14.5 wt %-15.5 wt %.

The mold has a negative Poisson's ratio structure, and in particular has a negative Poisson's ratio property under a strain condition of less than 50%. The negative Poisson's ratio property means that when subjected to tension, the material expands laterally within an elastic range, and when subjected to compression, the material contracts laterally.

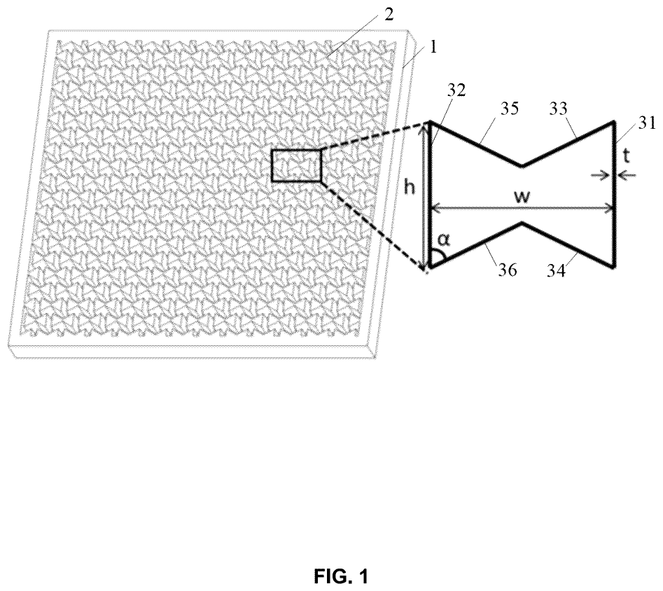

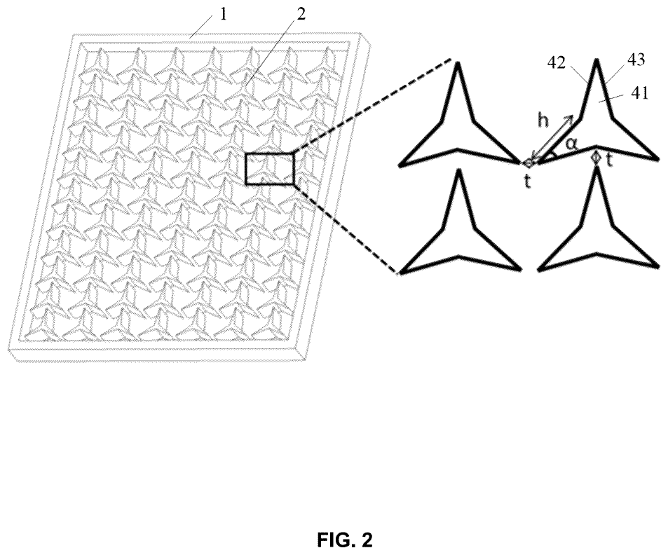

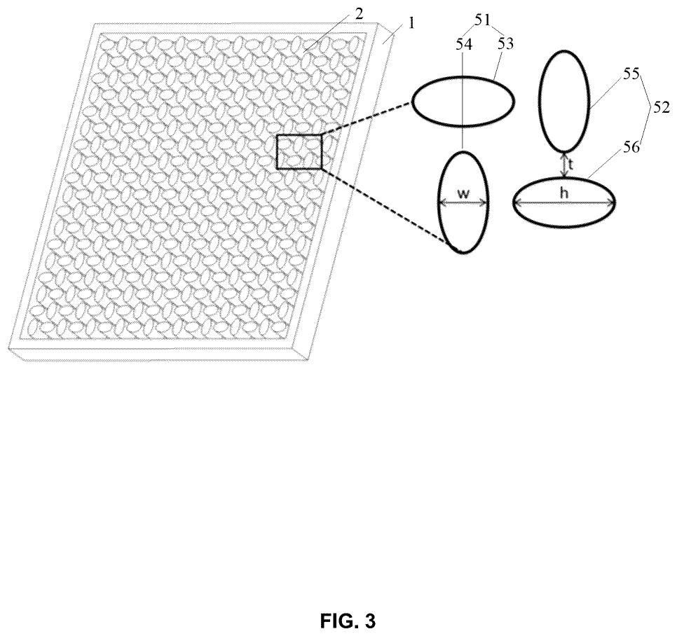

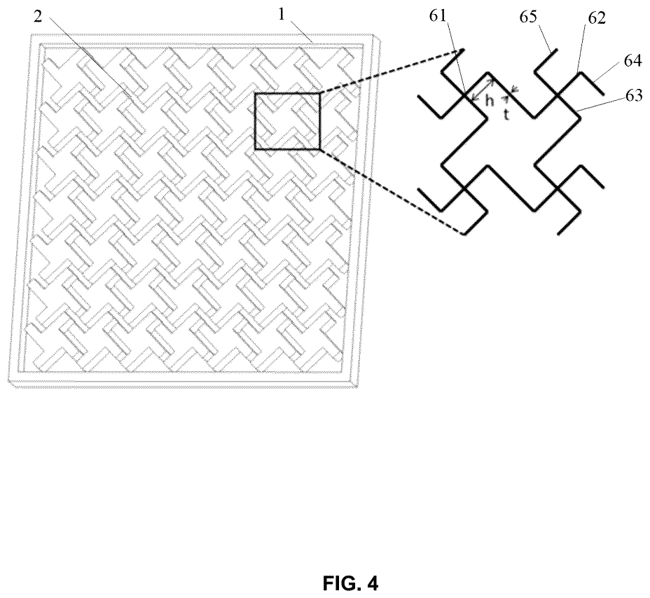

FIG. 1 is a schematic structural diagram illustrating an exemplary mold according to some embodiments of the present disclosure. FIG. 2 is a schematic structural diagram illustrating an exemplary mold according to some embodiments of the present disclosure. FIG. 3 is a schematic structural diagram illustrating an exemplary mold according to some embodiments of the present disclosure. FIG. 4 is a schematic structural diagram illustrating an exemplary mold according to some embodiments of the present disclosure. Referring to FIGS. 1-4, in some embodiments, the mold may include a border 1 and a plurality of periodically disposed unit elements 2. The plurality of unit elements 2 may be solid structures, and voids for filling the polyvinyl alcohol solution may be formed between adjacent unit elements 2, and between the unit elements 2 and the border 1. In some embodiments, the plurality of unit elements 2 may include at least one of a concave hexagonal unit element, a stellate unit element, a bend-induced unit element, and a chiral unit element.

Referring to FIG. 1, in some embodiments, each of the plurality of unit elements 2 may be the concave hexagonal unit element, and each of the plurality of unit elements 2 may include a bottom wall 31, a top wall 32, a first bottom sloping wall 33, a second bottom sloping wall 34, a first top sloping wall 35, and a second top sloping wall 36. In some embodiments, the bottom wall 31 and the top wall 32 may be arranged parallel to each other, the first bottom sloping wall 33 and the second bottom sloping wall 34 may be arranged on two sides of the bottom wall 31, and a first preset angle may be formed between the first bottom sloping wall 33 and the second bottom sloping wall 34 and the bottom wall 31. The first top sloping wall 35 and the second top sloping wall 36 may be arranged on two sides of the top wall 32, and a second preset angle may be formed between the first top sloping wall 35 and the second top sloping wall 36 and the top wall 32. One end of the first bottom sloping wall 33 may be connected with the bottom wall, the other end of the first bottom sloping wall 33 may be connected with the first top sloping wall 35. One end of the second bottom sloping wall 34 may be connected with the bottom wall 31, the other end of the second bottom sloping wall 34 may be connected with the second top sloping wall 36. The first bottom sloping wall 33 may be parallel to the second top sloping wall 36, and the second bottom sloping wall 34 may be parallel to the first top sloping wall 35. The unit elements 2 based on this structure have a relatively large negative Poisson's ratio deformation interval, and the penis enlargement implant obtained by molding can have a good fit with the deformation of the penis, so as to realize a high degree of compliance.

In some embodiments, a distance between the bottom wall 31 and the top wall 32, and a height of the bottom wall 31 may satisfy the following equation: tana ≥ w/h; where w denotes the distance between the bottom wall and the top wall, h denotes the height of the bottom wall, and a denotes the first preset angle or a second preset angle.

In some embodiments, the first preset angle and the second preset angle may be the same. For example, the first preset angle is in a range of 20°-60°, and the second preset angle is in a range of 20°-60°. As another example, the first preset angle is in a range of 25°-50°, and the second preset angle is in a range of 25°-50°. As another example, the first preset angle is in a range of 30°-45°, and the second preset angle is in a range of 30°-45°.

In some embodiments, the distance between the bottom wall 31 and the top wall 32 may be in a range of 1.8 mm-3.5 mm. For example, the distance between the bottom wall 31 and the top wall 32 is in a range of 2.0 mm-3.2 mm. As another example, the distance between the bottom wall 31 and the top wall 32 is in a range of 2.4 mm-3 mm.

In some embodiments, the height of the bottom wall 31 and the height of the top wall 32 may be the same in a range of 1 mm-4 mm. For example, the height of the bottom wall 31 and the height of the top wall 32 are in a range of 1.5 mm-3.5 mm. As another example, the height of the bottom wall 31 and the height of the top wall 32 are in a range of 2 mm-3 mm.

In some embodiments, distances between adjacent bottom walls and top walls, and distances between adjacent top sloping walls and bottom sloping walls of adjacent unit elements 2 are the same, respectively, such that each wall of the penis enlargement implant obtained by molding has the same thickness (referring to FIG. 1), i.e., the thickness of the bottom wall, the thickness of the top wall, the thickness of the first bottom sloping wall, the thickness of the second bottom sloping wall, the thickness of the first top sloping wall, and the thickness of the second top sloping wall are the same in a range of 0.6 mm-1.2 mm. For example, thickness of the bottom wall is in a range of 0.6 mm-1.2 mm, the thickness of the top wall is in a range of 0.6 mm-1.2 mm, the thickness of the first bottom sloping wall is in a range of 0.6 mm-1.2 mm, the thickness of the second bottom sloping wall is in a range of 0.6 mm-1.2 mm, the thickness of the first top sloping wall is in a range of 0.6 mm-1.2 mm, and the thickness of the second top sloping wall is in a range of 0.6 mm-1.2 mm. As another example, the thickness of the bottom wall is in a range of 0.7 mm-1.1 mm, the thickness of the top wall is in a range of 0.7 mm-1.1 mm, the thickness of the first bottom sloping wall is in a range of 0.7 mm-1.1 mm, the thickness of the second bottom sloping wall is in a range of 0.7 mm-1.1 mm, the thickness of the first top sloping wall is in a range of 0.7 mm-1.1 mm, and the thickness of the second top sloping wall is in a range of 0.7 mm-1.1 mm. As another example, the thickness of the bottom wall is in a range of 0.8 mm-1 mm, the thickness of the top wall is in a range of 0.8 mm-1 mm, the thickness of the first bottom sloping wall is in a range of 0.8 mm-1 mm, the thickness of the second bottom sloping wall is in a range of 0.8-1 mm, the thickness of the first top sloping wall is in a range of 0.8 mm-1 mm, and the thickness of the second top sloping wall is in a range of 0.8 mm-1 mm.

Referring to FIG. 2, In some embodiments, each of the plurality of unit elements 2 may be the stellate unit element, each of the plurality of unit elements 2 may be enclosed by three arrowhead units 41, and apex angles of the three arrowhead units 41 may be the same. For example, the apex angle of each of the three arrowhead units 41 is in a range of 10°-30°. As another example, the apex angle of each of the three arrowhead units 41 is in a range of 12°-25°. As another example, the apex angle of each of the three arrowhead units 41 is in a range of 15°-20°. In some embodiments, a distance between the adjacent unit elements 2 of the plurality of unit elements 2 may be in a range of 0.2 mm-1.0 mm. For example, the distance between the adjacent unit elements 2 of the plurality of unit elements 2 is in a range of 0.4 mm-0.9 mm. As another example, the distance between the adjacent unit elements 2 of the plurality of unit elements 2 is in a range of 0.5 mm-0.8 mm.

In some embodiments, the three arrowhead units 41 may be circumferentially distributed and evenly spaced. Each of the three arrowhead units 41 may include a first side 42 and a second side 43 which are connected with each other. In some embodiments, a length of the first side 42 and a length of the second side 43 may be the same in a range of 2 mm-4.5 mm. For example, the length of the first side 42 and the length of the second side 43 are in a range of 2.5 mm-4.2 mm. As another example, the length of the first side 42 and the length of the second side 43 are in a range of 3 mm-4 mm.

By circumferentially distributing and evenly spacing the three arrowhead units and making the first side and the second side have the same length, the unit elements can have better symmetry, which in turn makes the penis enlargement implant made from the mold based on this structure have a reasonable negative Poisson's ratio deformation interval in all directions.

In some embodiments of the present disclosure, by providing the unit elements as the stellate unit elements each of which enclosed by the three arrowhead units, the subsequent penis enlargement implant has a relatively large negative Poisson's ratio deformation interval, thereby providing a good fit with the deformation of the penis.

Referring to FIG. 3, In some embodiments, each of the plurality of unit elements 2 may be a bend-induced unit element. In some embodiments, each of the plurality of unit elements 2 may include a first unit 51 and a second unit 52. The first unit 51 may include a first ellipsoidal unit 53 of which a long axis set vertically and a second ellipsoidal unit 54 of which a long axis set horizontally. The second unit 52 may include a third ellipsoidal unit 55 of which a long axis is set vertically and a fourth ellipsoidal unit 56 of which a long axis is set horizontally. The long axis of the first ellipsoidal unit 53 and the long axis of the fourth ellipsoidal unit 56 may be parallel to each other, and the long axis of the second ellipsoidal unit 54 and the long axis of the third ellipsoidal unit 55 may be parallel to each other. The long axis of the first ellipsoidal unit 53 may be coaxial with a short axis of the third ellipsoidal unit 55, and a short axis of the second ellipsoidal unit 54 may be coaxial with the long axis of the fourth ellipsoidal unit 56.

In some embodiments, lengths of the long axes of the first ellipsoidal unit 53, the second ellipsoidal unit 54, the third ellipsoidal unit 55, and the fourth ellipsoidal unit 56 may be the same, and lengths of the short axes of the first ellipsoidal unit 53, the second ellipsoidal unit 54, the third ellipsoidal unit 55, and the fourth ellipsoidal unit 56 may be the same. In some embodiments, a length w of the long axis may be in a range of 1.5 mm-5.5 mm. For example, the length w of the long axis is in a range of 2.0 mm-5.0 mm. As another example, the length w of the long axis is in a range of 2.5 mm-4.5 mm. In some embodiments, a length h of the short axis may be in a range of 0.5 mm-3.5 mm. For example, the length h of the short axis is in a range of 1 mm-3 mm. As another example, the length h of the short axis is in a range of 1.5 mm-2.5 mm.

By making the long axes of the first ellipsoidal unit, the second ellipsoidal unit, the third ellipsoidal unit, and the fourth ellipsoidal unit have the same length, and the short axes of the first ellipsoidal unit, the second ellipsoidal unit, the third ellipsoidal unit, and the fourth ellipsoidal unit have the same length, the elliptical units having the appropriate sizes and the same lengths of long axes and short axes can be arranged reasonably.

In some embodiments, a distance t between the first ellipsoidal unit 53 and the second ellipsoidal unit 54 is the same as a distance between the first ellipsoidal unit 53 and the third ellipsoidal unit 55 in a range of 0.1 mm-1.5 mm. For example, the distance t is in a range of 0.3 mm-1.2 mm. As another example, the distance t is in a range of 0.5 mm-1.0 mm.

By making the distance between the first ellipsoidal unit and the second ellipsoidal unit the same as the distance between the first ellipsoidal unit and the third ellipsoidal unit, different ellipsoidal units can be evenly distributed, such that the penis enlargement implant molded through the mold of this structure has more stable mechanical properties.

In some embodiments of the present disclosure, the penis enlargement implant molded through the mold of this structure has anisotropy in the vertical and horizontal directions by making the ellipsoidal units of the first unit and the second unit have different settings in the direction of the long axis, which makes the penis enlargement implant molded by the mold of this structure have strong mechanical properties, and makes the penis enlargement implant change along with the deformation of the penis, thereby realizing higher degree of fit.

Referring to FIG. 4, In some embodiments, each of the plurality of the unit elements 2 may be a chiral structural unit element, and each of the plurality of unit elements may include a fylfot-shaped unit 61. The fylfot-shaped unit 61 may include a first wall 62 and a second wall 63 which are perpendicular to each other. Each of two ends of the first wall 62 may be provided with a first bending portion 64 perpendicular to the first wall 62, and each of two ends of the second wall 63 may be provided with a second bending portion 65 perpendicular to the second wall 63. In some embodiments, each of the unit elements 2 may include four fylfot-shaped units 61. The first bending portions 64 and the second bending portions 65 of adjacent fylfot-shaped units 61 may be connected.

In some embodiments, a thickness t of the first wall 62, a thickness t of the second wall 63, a thickness t of the first bending portion 64, and a thickness t of the second bending portion 65 may be the same in a range of 0.1 mm-1.5 mm. For example, the thickness t is in a range of 0.8 mm-1.2 mm. As another example, the thickness t is in a range of 0.9 mm-1.1 mm.

By making the first wall, the second wall, the first bending portion, and the second bending portion have the same thickness, better arrangement and connection between a plurality of fylfot-shaped units can be achieved, and the penis enlargement implant molded through the mold of this structure has better mechanical properties so as to well fit the deformation of the penis.

In some embodiments, a distance h between the first bending portion 64 and the second wall 63 may be the same as a distance between a second bending portion 65 and the first wall 62 in a range of 0.5 mm-1.5 mm. Fr example, the distance h is in a range of 0.3 mm-1.2 mm. As another example, the distance h is in a range of 0.5 mm-2 mm.

By setting the distance between the first bending portion and the second wall to be the same as the distance between the second bending portion and the first wall, the plurality of fylfot-shaped units can be evenly arranged, thereby making the mechanical properties of the unit elements more balanced in all directions.

In some embodiments, the above mold may be prepared using a 3D printing molding process, which in turn may be customized to meet individual needs.

S2: Performing at least three freeze-thaw cycles on the mold and the polyvinyl alcohol solution to obtain a hydrogel sample.

In some embodiments, the mold and the polyvinyl alcohol solution are frozen at a temperature from −30° C. to −15° C. for 6 h-12 h, then thawed at a temperature from 10° C. to 40° C. for 4 h-6 h, and the freezing and the thawing are cycled for 2-5 times to obtain the hydrogel sample. In some embodiments, the mold and the polyvinyl alcohol solution are frozen at a temperature from −30° C. to −18° C. for 6 h-8 h, then thawed at a temperature from 30° C. to 40° C. for 4 h-5 h, and the freezing and the thawing are cycled for 3-4 times to obtain the hydrogel sample. In some embodiments, the mold and the polyvinyl alcohol solution are frozen at a temperature from −25° C. to −15° C. for 8 h-11 h, then thawed at a temperature from 10° C. to 25° C. for 5 h-6 h, and the freezing and the thawing are cycled for 4-5 times to obtain the hydrogel sample.

The hydrogel sample is demolded after cyclic freezing and thawing. The hydrogel sample after demolding has a preset length, which is conducive to fixing and suturing to the penis. The preset length is an initial length of the hydrogel sample in a state where the hydrogel sample is not stretched, and is related to the length of the mold. In some embodiments, the preset length may include a plurality of manually preset lengths. In some embodiments, a plurality of hydrogel samples may be molded according to specific needs, a count of structural units in the plurality of hydrogel samples corresponding to the length and girth of the penis required to be implanted when the penis is flaccid.

In some embodiments of the present disclosure, by performing at least three freeze-thaw cycles on the mold and the polyvinyl alcohol solution therein, physical cross-linking between molecular chains of the polyvinyl alcohol can be induced and the count of cross-linking points can be increased. The increased cross-linking points make the network structure of the hydrogel sample more stable, thereby improving the mechanical properties of the hydrogel sample.

S3: Soaking the hydrogel sample in a lye for a preset time period, and then washing to remove impurities to obtain the penis enlargement implant.

In some embodiments, the lye includes but is not limited to a NaOH solution or a KOH solution. A concentration of the lye may be in a range of 17.5 wt %-22.5 wt %. For example, the concentration of the lye is in a range of 18 wt %-22 wt %. As another example, the concentration of the lye is in a range of 19 wt %-21 wt %. As another example, the concentration of the lye is in a range of 19.5 wt %-20.5 wt %. The crystallinity of the hydrogel sample can be enhanced by soaking in the lye, thereby further enhancing the mechanical properties and stability. In some embodiments, after soaking in the lye, an elongation at break of the hydrogel sample is greater than or equal to 200% (e.g., 200%-400%), a Young's modulus of the hydrogel sample is greater than or equal to 1 MPa, and a compression modulus of the hydrogel sample is greater than or equal to 200 kPa, which can effectively satisfy the needs of use.

In some embodiments, the hydrogel sample is placed in the lye and stirred to crystallize for 20 h-30 h, and then placed in water (which may be deionized water) and stirred to clean for 22 h-26 h to obtain the penis enlargement implant. During the stirring and cleaning, the water is changed every 4 h-8 h. In some embodiments, the hydrogel sample is placed in a NaOH solution and stirred to crystallize for 25 h-30 h, and then placed in deionized water and stirred to clean for 24 h-25 h to obtain the penis enlargement implant. During the stirring and cleaning, the water is changed every 6 h-8 h. In some embodiments, the hydrogel sample is placed in a KOH solution and stirred to crystallize for 22 h-26 h, and then placed in deionized water and stirred to clean for 22 h-24 h to obtain the penis enlargement implant. During the stirring and cleaning, the water is changed every 4 h-6 h.

The preparation method provided by the embodiments of the present disclosure uses a biocompatible hydrogel as a matrix material, which is not easily degraded and can be used as a long-term implant. The hydrogel sample obtained by the preparation method of the embodiments of the present disclosure has the elongation at break of 200%-400% and the mechanical properties matching the tissue properties after soaking in the lye, and has good adaptability during penile erection.

The technical solutions provided by the present disclosure are described in detail below with reference to the embodiments, but are not to be construed as limiting the scope of protection of the present disclosure.

### Embodiment 1

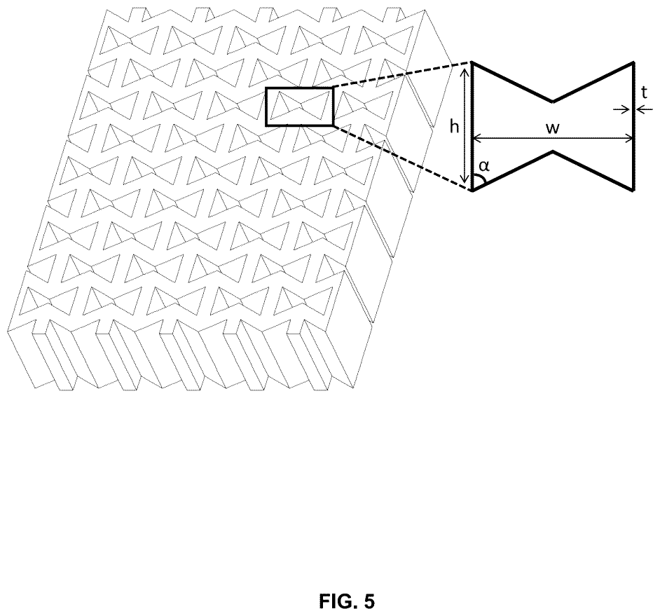

FIG. 5 is a schematic structural diagram illustrating an exemplary penis enlargement implant prepared according to Embodiment 1 of the present disclosure.

Modeling is performed using the modeling software to construct a model of a concave hexagonal structural mold with a negative Poisson's ratio structure referring to FIG. 1, where a length h of the model is 2 mm, a length w of the model is 2.4 mm, a pitch t of the model is 0.8 mm, and an angle a of the model is 60°. The model is saved in the stl format after the model is constructed.

The designed mold is printed based on the model using commercial photosensitive resin (e.g., eSUN PMMA acrylic sheet resin) as a raw material and a high-precision DLP printer with a photo resolution of 15 μm, a printing speed of 2.5 s/layer, a printing power of 250 W, and a height per layer of 50 μm. The printed mold is put into a curing box and cured at a condition of 405 nm for 3 min to obtain a cured mold.

15 g of polyvinyl alcohol (PVA) with a molecular weight of 146000 Da-186000 Da is added to deionized water and dissolved in a water bath at 90° C. for 2 h with vigorous stirring to obtain a PVA solution with a mass fraction of 15 wt %. The PVA solution is loaded into mold voids, placed in a vacuum oven, evacuated to remove air bubbles in the PVA solution, scraped flat using a glass slide, placed in an environment of −20° C. to freeze for 8 h, then taken out and thawed at the room temperature for 4 h. The freezing-thawing cycles are repeated 3 times to obtain a PVA hydrogel with a negative Poisson's ratio structure. The obtained PVA hydrogel is soaked in sodium hydroxide (NaOH) solution with a mass fraction of 20 wt % for 24 h and slowly stirred, taken out and placed in deionized water for 24 h and slowly stirred with the deionized water being replaced every 8 h to obtain the penis enlargement implant.

The schematic diagram of the penis enlargement implant prepared according to Embodiment 1 is found in FIG. 5.

### Embodiment 2

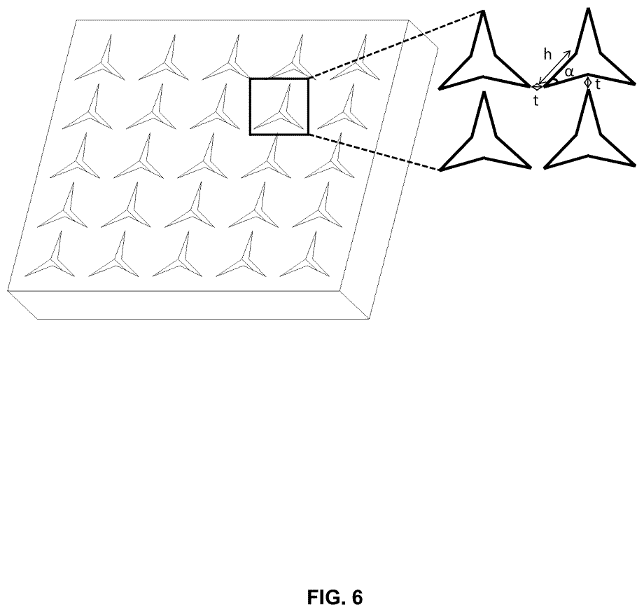

FIG. 6 is a schematic structural diagram illustrating an exemplary penis enlargement implant prepared according to Embodiment 2 of the present disclosure.

Embodiment 2 is different from Embodiment 1 in that the prepared mold with the negative Poisson's ratio structure is a stellate mold, referring to FIG. 2, the length h is 3 mm, the pitch t is 0.5 mm, and the angle a is 15°. The schematic diagram of the penis enlargement implant prepared according to Embodiment 2 is found in FIG. 6.

### Embodiment 3

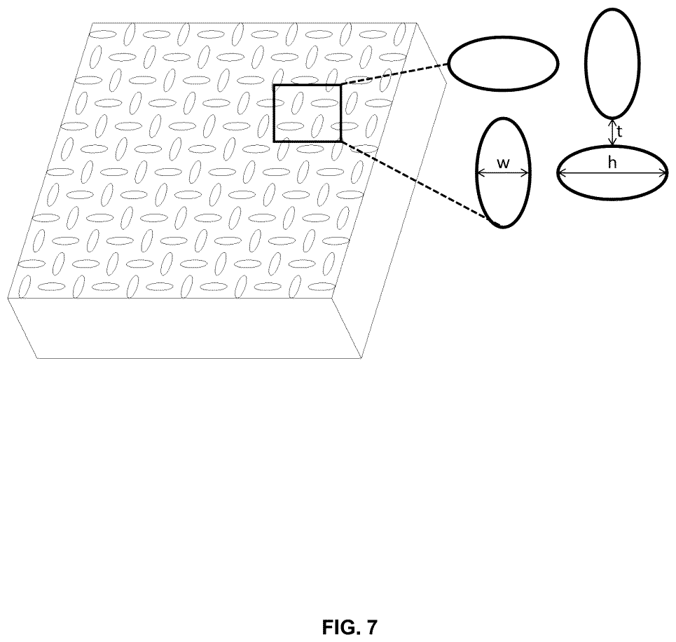

FIG. 7 is a schematic structural diagram illustrating an exemplary penis enlargement implant prepared according to Embodiment 3 of the present disclosure.

The Embodiment 3 is different in Embodiment 1 in that the prepared mold with the negative Poisson's ratio structure is a bend-induced mold, referring to FIG. 3, the length h is 4.5 mm, the width is 2.5 mm, and the pitch t is 1 mm. The schematic diagram of the penis enlargement implant prepared according to Embodiment 3 is found in FIG. 7.

### Embodiment 4

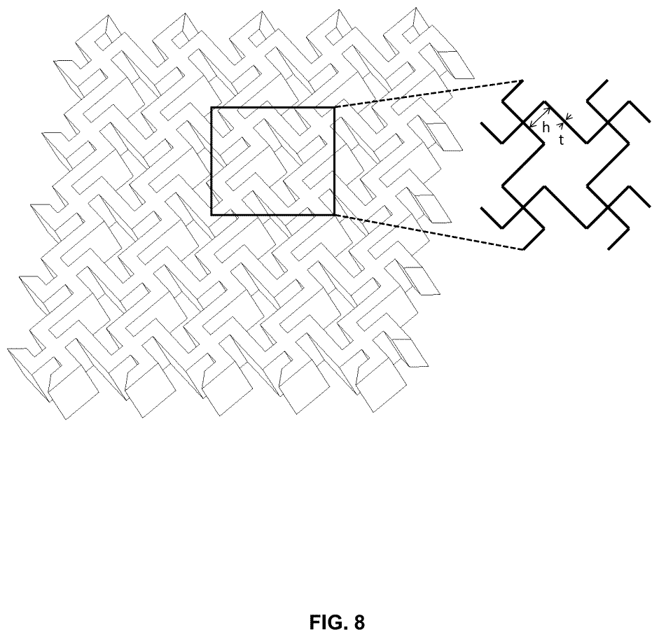

FIG. 8 is a schematic structural diagram illustrating an exemplary penis enlargement implant prepared according to Embodiment 4 of the present disclosure.

Embodiment 4 is different from Embodiment 1 in that the prepared mold with the negative Poisson's ratio structure is a chiral mold, referring to FIG. 4, the length h is 0.5 mm, and the pitch t is 1 mm. The schematic diagram of the penis enlargement implant prepared according to Embodiment 4 is found in FIG. 8.

### Contrast Embodiment 1

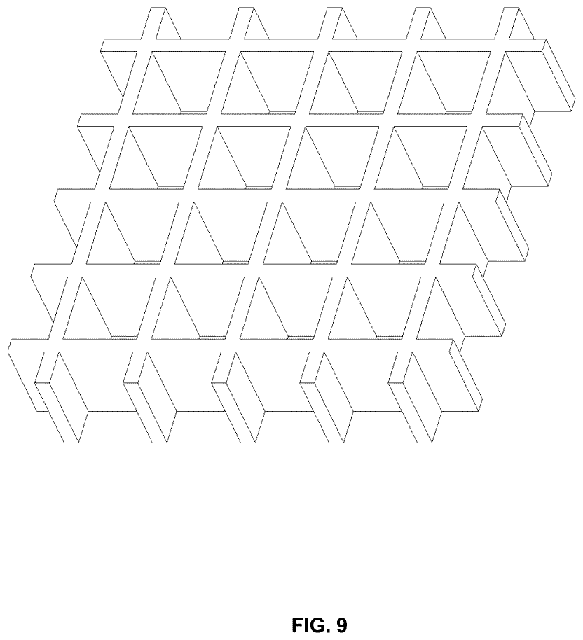

FIG. 9 is a schematic structural diagram illustrating an exemplary penis enlargement implant prepared according to Contrast Embodiment 1 of the present disclosure.

The Contrast Embodiment 1 is different from Embodiment 1 in that the structure of the penis enlargement implant prepared based on the mold of the Contrast Embodiment 1 is a square structure of FIG. 9.

### Contrast Embodiment 2

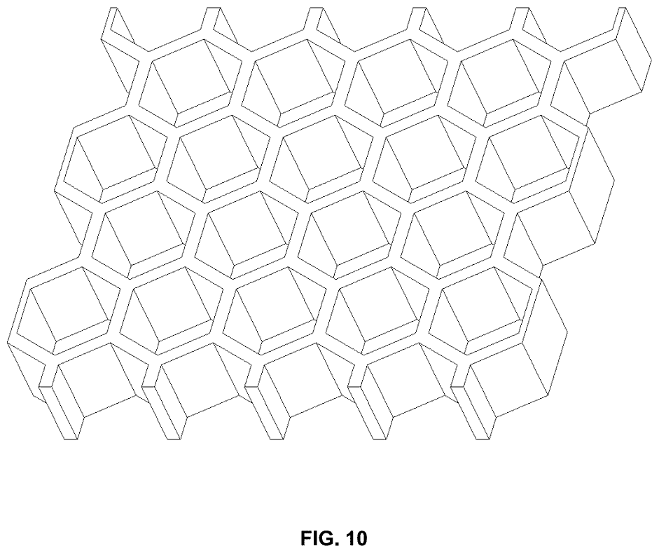

FIG. 10 is a schematic structural diagram illustrating an exemplary penis enlargement implant prepared according to Contrast Embodiment 2 of the present disclosure.

The Contrast Embodiment 2 is different from Embodiment 1 in that the structure of the penis enlargement implant prepared based on the mold of the Contrast Embodiment 2 is a honeycomb structure of FIG. 10.

### Contrast Embodiment 3

The Contrast Embodiment 3 is different from Embodiment 1 in that the PVA is not soaked in the NaOH solution.

### Contrast Embodiment 4

The Contrast Embodiment 4 is different from Embodiment 1 in that the material used is gelatin methacrylate (GelMA). 15 g commercial GelMA is dissolved in deionized water in a water bath at 37° C. for 2 h and vigorously stirred to obtain a GelMA solution with a mass fraction of 15 wt %. The GelMA solution is loaded into the mold of Embodiment 1, placed in a vacuum oven, evacuated to remove air bubbles in the GelMA solution, scraped flat using a glass slide, placed under a UV lamp and irradiated for 2 min to obtain the GelMA hydrogel with the negative Poisson's ratio structure as the penis enlargement implant.

The penis enlargement implants prepared according to Embodiments 1˜4 and Contrast Embodiments 1˜4 are tested and evaluated, and the results are shown below.

According to the Embodiments 1-4, with the design of the negative Poisson's ratio structure and the use of PVA hydrogel soaked in the NaOH solution for enhancement, the prepared penis enlargement implants deform during penis erection with simultaneous material stretching, the tensile deformation can reach 1.5-2 times the original size, and the penis enlargement implants can maintain the thickened shape of the penis after implantation without significant thickness change (>5%) with penile erection and flaccidity. Moreover, the elongation at break of the matrix material is greater than the size change of penile erection, thereby satisfying the implantation requirements.

According to the Contrast Embodiments 1-2, due to the lack of the design of the negative Poisson's ratio structure, the material is stretched to deform under tension, leading to a greater stress on the penis and forming a burden.

For the penis enlargement implant prepared according to the Contrast Embodiment 3, the strength is lower and more unstable due to the lack of soaking in the NaOH solution to enhance the crystalline zone; after implantation, it is difficult to maintain the thickened shape, resulting in significant thickness change during penile erection. In addition, due to the reduced crystalline zone, long-term implantation may lead to gradual dissolution of the PVA hydrogel in body fluids, which further decreases the strength of the penis enlargement implant, resulting in failure of the enlargement function.

The penis enlargement implant prepared according to Contrast Embodiment 4 has significantly weaker mechanical properties than the PVA hydrogel enhanced by soaking in lye. After implantation, it is difficult to maintain the thickened shape during penis erection. Long-term implantation may lead to gradual dissolution of the PVA hydrogel in body fluids, which further decreases the mechanical strength of the penis enlargement implant, resulting in failure of the enlargement function.

According to some embodiments of the present disclosure, four negative Poisson's ratio structures that can be applied to the penis enlargement implant are provided, and the penis enlargement implant obtained based on the structures has systole and diastole that can fit well with the deformation of the penis and can be well used as a penile enlargement patch.

As can be seen from the above embodiments, the penis enlargement implant and the preparation method thereof provided in the present disclosure use the biocompatible hydrogel as the matrix material, and the mechanical properties match the penile tissues, and the safety and stability are good. The design of the negative Poisson's ratio structure is arranged, which can realize the negative Poisson's ratio effect and anisotropy under low strain, and realize that the structure changes first and the material is stretched later during the change with the penis erection, thereby realizing the lightweight and sports-adapted properties of the penis enlargement implant. With the 3D printing mold structure, personalization for the patient can be achieved, and the penis enlargement implant is easy to prepare and short in preparation cycle.

In addition, the size of the penis enlargement implant obtained by the preparation method provided by the embodiments of the present disclosure can be adjusted, and the size and thickness of the penis enlargement implant, and the deformation interval of the negative Poisson's ratio can be determined according to the actual condition of the patient, which can realize customization for the patient, short preparation cycle, and low cost.

The above disclosure is only one of the preferred embodiments of the present disclosure, and cannot be used to limit the scope of the claims of the present disclosure, and therefore equivalent changes made according to the claims of the present disclosure are still covered by the scope of the present disclosure.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure, or feature described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or features may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various parts described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, numbers describing the number of ingredients and attributes are used. It should be understood that such numbers used for the description of the embodiments use the modifier “about”, “approximately”, or “substantially” in some examples. Unless otherwise stated, “about”, “approximately”, or “substantially” indicates that the number is allowed to vary by +20%. Correspondingly, in some embodiments, the numerical parameters used in the description and claims are approximate values, and the approximate values may be changed according to the required features of individual embodiments. In some embodiments, the numerical parameters should consider the prescribed effective digits and adopt the method of general digit retention. Although the numerical ranges and parameters used to confirm the breadth of the range in some embodiments of the present disclosure are approximate values, in specific embodiments, settings of such numerical values are as accurate as possible within a feasible range.

For each patent, patent application, patent application publication, or other materials cited in the present disclosure, such as articles, books, specifications, publications, documents, or the like, the entire contents of which are hereby incorporated into the present disclosure as a reference. The application history documents that are inconsistent or conflict with the content of the present disclosure are excluded, and the documents that restrict the broadest scope of the claims of the present disclosure (currently or later attached to the present disclosure) are also excluded. It should be noted that if there is any inconsistency or conflict between the description, definition, and/or use of terms in the auxiliary materials of the present disclosure and the content of the present disclosure, the description, definition, and/or use of terms in the present disclosure is subject to the present disclosure.

Finally, it should be understood that the embodiments described in the present disclosure are only used to illustrate the principles of the embodiments of the present disclosure. Other variations may also fall within the scope of the present disclosure. Therefore, as an example and not a limitation, alternative configurations of the embodiments of the present disclosure may be regarded as consistent with the teaching of the present disclosure. Accordingly, the embodiments of the present disclosure are not limited to the embodiments introduced and described in the present disclosure explicitly.

What is claimed is:

1. A preparation method of a penis enlargement implant, comprising:
loading a polyvinyl alcohol solution into a mold, the mold having a negative Poisson's ratio structure;
performing at least three freeze-thaw cycles on the mold and the polyvinyl alcohol solution to obtain a hydrogel sample; and
soaking the hydrogel sample in a lye for a preset time period, and then washing to remove impurities to obtain the penis enlargement implant.

2. The preparation method of claim 1, wherein a molecular weight of polyvinyl alcohol in the polyvinyl alcohol solution is in a range of 10000 Da-196000 Da, and an alcoholysis degree is in a range of 90%-98%.

3. The preparation method of claim 1, wherein a concentration of the polyvinyl alcohol solution is in a range of 12.5 wt %-17.5 wt %.

4. The preparation method of claim 1, wherein the lye is a NaOH solution or a KOH solution, and a concentration of the NaOH solution or a KOH solution is in a range of 17.5 wt %-22.5 wt %.

5. The preparation method of claim 1, wherein an elongation at break of the hydrogel sample is greater than or equal to 200%, a Young's modulus of the hydrogel sample is greater than or equal to 1 MPa, and a compression modulus of the hydrogel sample is greater than or equal to 200 kPa.

6. The preparation method of claim 1, wherein the performing at least three freeze-thaw cycles on the mold and the polyvinyl alcohol solution to obtain a hydrogel sample includes: freezing the mold and the polyvinyl alcohol solution at a temperature from −30° C. to −15° C. for 6 h-12 h, then thawing at a temperature from 10° C. to 40° C. for 4 h-6 h, and cycling the freezing and the thawing for 3-5 times to obtain the hydrogel sample.

7. The preparation method of claim 1, wherein the soaking the hydrogel sample in a lye for a preset time period, and then washing to remove impurities to obtain the penis enlargement implant includes: placing the hydrogel sample in the lye and stirring for 20 h-30 h, and then placing the hydrogel sample in water and stirring and washing for 22 h-26 h to obtain the penis enlargement implant, during the stirring and the washing, changing the water every 4 h-8 h.

8. The preparation method of claim 1, wherein the mold includes a border and a plurality of unit elements periodically arranged;
the plurality of unit elements include at least one of a concave hexagonal unit element, a stellate unit element, a bend-induced unit element, and a chiral unit element.

9. The preparation method of claim 8, wherein each of the plurality of unit elements is the concave hexagonal unit element, and each of the plurality of unit elements includes a bottom wall, a top wall, a first bottom sloping wall, a second bottom sloping wall, a first top sloping wall, and a second top sloping wall;
the bottom wall and the top wall are arranged parallel to each other, the first bottom sloping wall and the second bottom sloping wall are arranged on two sides of the bottom wall, and a first preset angle is formed between the first bottom sloping wall and the second bottom sloping wall and the bottom wall; the first top sloping wall and the second top sloping wall are arranged on two sides of the top wall, and a second preset angle is formed between the first top sloping wall and the second top sloping wall and the top wall;
one end of the first bottom sloping wall is connected with the bottom wall, the other end of the first bottom sloping wall is connected with the first top sloping wall, one end of the second bottom sloping wall is connected with the bottom wall, the other end of the second bottom sloping wall is connected with the second top sloping wall, the first bottom sloping wall is parallel to the second top sloping wall, and the second bottom sloping wall is parallel to the first top sloping wall.

10. The preparation method of claim 9, wherein a distance between the bottom wall and the top wall, and a height of the bottom wall satisfy the following equation:
tana ≥w/h;
where w denotes the distance between the bottom wall and the top wall, h denotes the height of the bottom wall, and a denotes the first preset angle.

11. The preparation method of claim 9, wherein the first preset angle and the second preset angle are the same; the first preset angle is in a range of 20°-60°, and the second preset angle is in a range of 20°-60°.

12. The preparation method of claim 9, wherein the distance between the top wall and the bottom wall is in a range of 1.8 mm-3.5 mm.

13. The preparation method of claim 9, wherein a height of the bottom wall and a height of the top wall are the same; the height of the bottom wall is in a range of 1 mm-4 mm, and the height of the top wall is in a range of 1 mm-4 mm.

14. The preparation method of claim 9, wherein a thickness of the bottom wall, a thickness of the top wall, a thickness of the first bottom sloping wall, a thickness of the second bottom sloping wall, a thickness of the first top sloping wall, and a thickness of the second top sloping wall are the same; the thickness of the bottom wall is in a range of 0.6 mm-1.2 mm, the thickness of the top wall is in a range of 0.6 mm-1.2 mm, the thickness of the first bottom sloping wall is in a range of 0.6 mm-1.2 mm, the thickness of the second bottom sloping wall is in a range of 0.6 mm-1.2 mm, the thickness of the first top sloping wall is in a range of 0.6 mm-1.2 mm, and the thickness of the second top sloping wall is in a range of 0.6 mm-1.2 mm.

15. The preparation method of claim 8, wherein each of the plurality of unit elements is the stellate unit element, each of the plurality of unit elements is enclosed by three arrowhead units, apex angles of the three arrowhead units are the same, and the apex angle of each of the three arrowhead units is in a range of 10°-30°; and/or a distance between adjacent unit elements of the plurality of unit elements is in a range of 0.2 mm-1.0 mm.

16. The preparation method of claim 8, wherein each of the plurality of unit elements is the bend-induced unit element, and each of the plurality of unit elements includes a first unit and a second unit; the first unit includes a first ellipsoidal unit of which a long axis is set vertically and a second ellipsoidal unit of which a long axis is set horizontally; the second unit includes a third ellipsoidal unit of which a long axis is set vertically and a fourth ellipsoidal unit of which a long axis is set horizontally; the long axis of the first ellipsoidal unit and the long axis of the fourth ellipsoidal unit are parallel to each other, the long axis of the second ellipsoidal unit and the long axis of the third ellipsoidal unit are parallel to each other; the long axis of the first ellipsoidal unit is coaxial with a short axis of the third ellipsoidal unit, and a short axis of the second ellipsoidal unit is coaxial with the long axis of the fourth ellipsoidal unit.

17. The preparation method of claim 16, wherein a length of the long axis of the first ellipsoidal unit, a length of the long axis of the second ellipsoidal unit, a length of the long axis of the third ellipsoidal unit, and a length of the long axis of the fourth ellipsoidal unit are the same, and a length of the short axis of the first ellipsoidal unit, a length of the short axis of the second ellipsoidal unit, a length of the short axis of the third ellipsoidal unit, and a length of the short axis of the fourth ellipsoidal unit are the same; the length of the long axis is in a range of 1.5 mm-5.5 mm, and the length of the short axis is in a range of 0.5 mm-3.5 mm; and/or
a distance between the first ellipsoidal unit and the second ellipsoidal unit is the same as a distance between the first ellipsoidal unit and the third ellipsoidal unit; the distance between the first ellipsoidal unit and the second ellipsoidal unit is in a range of 0.1 mm-1.5 mm, and the distance between the first ellipsoidal unit and the third ellipsoidal unit is in a range of 0.1 mm-1.5 mm.

18. The preparation method of claim 8, wherein each of the plurality of unit elements is the chiral structural unit element, and each of the plurality of unit elements includes a fylfot-shaped unit, and the fylfot-shaped unit includes a first wall and a second wall which are perpendicular to each other; each of two ends of the first wall is provided with a first bending portion perpendicular to the first wall, and each of two ends of the second wall is provided with a second bending portion perpendicular to the second wall.

19. The preparation method of claim 18, wherein a thickness of the first wall, a thickness of the second wall, a thickness of the first bending portion, and a thickness of the second bending portion are the same, and the thickness of the first wall, the thickness of the second wall, the thickness of the first bending portion, and the thickness of the second bending portion are in a range of 0.1 mm-1.5 mm, respectively;
a distance between the first bending portion and the second wall is the same as a distance between the second bending portion and the first wall; the distance between the first bending portion and the second wall is in a range of 0.5 mm-1.5 mm, and the distance between the second bending portion and the first wall is in a range of 0.5 mm-1.5 mm.

20. A penis enlargement implant, prepared by the preparation method of claim 1.
